(19) United States
(12) Patent Application Publication
(10) Pub. No.: US 2023/0360161 A1
(43) Pub. Date: Nov. 9, 2023

(54) MANAGING WORKER SAFETY TO COMBAT FATIGUE

(71) Applicant: Kyndryl, Inc., New York, NY (US)

(72) Inventors: Prasanna Kumar DIXIT J, Bangalore (IN); Sharat K. TRIPATHY, Bangalore (IN)

(21) Appl. No.: 17/736,328

(22) Filed: May 4, 2022

(51) Int. Cl. G06Q 50/26; A61B 5/16; A61B 5/00; A61B 5/024; A61B 5/0533

(52) U.S. Cl. CPC G06Q 50/265; A61B 5/165; A61B 5/7267; A61B 5/02438; A61B 5/02416; A61B 5/02405; A61B 5/0533; A61B 2503/20

(57) ABSTRACT

A computer-implemented method includes: receiving, by a computing device, data which is associated with a user; generating, by the computing device, a personalized recommendation for the data by at least one artificial intelligence (AI) application; training, by the computing device, a machine learning (ML) model using the data from the at least one AI application; and generating, by the computing device, a trained fitness model for predicting safety issues based on the trained ML model using the data from the at least one AI application.

### BACKGROUND

Aspects of the present invention relate generally to a system and method to ensure worker safety and, more particularly, to a system and method to combat fatigue and other worker safety issues using sensors, wearables, and contextual behavioral health remediation to remotely monitor worker safety and support continuous operations in wireless applications.

Many workers are exposed to fatigue risk both on the job and away from the job. Worker fatigue may result in increased sick time and absenteeism, decreased attention, impaired focus, and irritability. Frameworks for managing hazards and risk in workplaces axe applicable to occupational fatigue and have spurred the development of new approaches for fatigue.

### SUMMARY

In a first aspect of the invention, there is a computer-implemented method including: receiving, by a computing device, data which is associated with a user; generating, by the computing device, a personalized recommendation for the data by at least one artificial intelligence (AI) application; training, by the computing device, a machine learning (ML) using the data from the at least one AI application; and generating, by the computing device, a trained fitness model for predicting safety issues based on the trained ML model using the data from at least one AI application.

In another aspect of the invention, there is a computer program product including one or more computer readable storage media having program instructions collectively stored on the one or more computer readable storage media. The program instructions are executable to: receive, at a computing system, wearable device (WD) data of a user from a data acquisition system at an edge node cluster; determine, at the computing system, whether the user has a safety issue by comparing the WD data and previous health pattern data; determine, at the computing system, whether a duration of the safety issue based on the WD data is greater than a predetermined threshold in response to a determination that the user has the safety issue; and automatically provide a worker safety recommendation alert, at the computing system, in response to a determination that the duration of the safety issue based on the WD data is greater than the predetermined threshold.

In another aspect of the invention, there is system including a processor, a computer readable memory, one or more computer readable storage media, and program instructions collectively stored on the one or more computer readable storage media. The program instructions are executable to: receive data which is associated with fatigue at an edge node cluster; generate a personalized recommendation to limit worker safety issues for the data associated with the fatigue by at least one artificial intelligence (AI) application; train a machine leaning (ML) model using the data from at least one AI application and historical information associated with the data; and generate a trained fitness model which predicts safety issues based on the trained ML model using the data from the at least one AI application and the historical information associated with the data.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a cloud computing node according to an embodiment of the present invention.

FIG. 2 depicts a cloud computing environment according to an embodiment of the present invention.

FIG. 3 depicts abstraction model layers according to an embodiment of the present invention.

FIG. 4 shows a block diagram for fatigue identification and behavioral remediation in accordance with aspects of the invention.

FIG. 5 shows a flowchart of a fatigue tracking deep learning model according to an embodiment of the present invention.

FIG. 6 shows sample model training data for the fatigue tracking learning model according to an embodiment of the present invention.

FIG. 7 shows a flowchart of fatigue identification in accordance with aspects of the invention.

FIG. 8 shows a sample of wearable device data for the fatigue identification according to an embodiment of the present invention.

FIG. 9 shows a sample of predicted fatigue data for the fatigue identification according to an embodiment of the present invention.

FIGS. 10 and 11 show graphs of predicted fatigue periods in accordance with aspects of the invention.

### DETAILED DESCRIPTION

Aspects of the present invention relate generally to a system and method to ensure worker safety and, more particularly, to a system and method to combat fatigue and other worker safety issues using sensors, wearables, and contextual behavioral health remediation to remotely monitor worker safety and support continuous operations using wireless applications. In an example, the systems and methods described herein rely on devices and pertinent data to manage workplace fatigue or monitor and remediate safety issues. These systems and methods allow supervisors, for example, to immediately recognize the impact of sleep deprivation, exposure to chemicals or other health issues, etc., which may have a direct impact on an individual's ability to perform work at a high level and in a safe manner.

Aspects of the present invention include a system, method and computer program product which allows system integrators the capability to combat fatigue or other safety issues using contextual behavioral remediation, and to manage continuous operation services in highly scalable critical mobile and edge applications. For example, the system, method and computer program product enables a transformation in worker behavior and a risk management plan for remediation. For example, aspects of the present invention may control radiofrequency electromagnetic field (EMF) exposure, provide artifacts related to exposure of substances in environments and employ exposure durations sufficient to assess disease processes, and provide an intelligence to build evidence to study patterns of adverse health effects of a worker for exposure levels of certain substances, for example. Aspects of the present invention also include a system, method and computer program product to subscribe to a fatigue tracking system, to proactively alert/warn a manager about potential risks, to provide auditable artifacts for safety and security compliance management, and to combine patterns between human behavior responses with genomic traits and other traits (e.g., skill level, experience, regular physical activities, etc.). For example, the auditable artifacts may include information which compares a current environment to exposure environments and/or information related to exposure durations which are sufficient to assess disease processes.

Aspects of the present invention may also assist operation teams in assessing individual strength, fatigue assessment scales, need for recovery, and occupational fatigue exhaustion recovery. Aspects of the present invention may also reduce the dependency on leveraging stressed skilled resources for mission critical applications, assist in reducing the risk of skilled resources becoming depressed, and for upskilling and talent retention. In embodiments, by implementing the system, method and computer program product herein, remediation insights may be available in real time and in a continuous operational model to manage mobile and edge device information, which is a building block to distribute the workload deployed across a network cloud/edge data center. Also, aspects of the present invention may assist in improving the security operations by enforcing audit compliance and managing risk effectively, implement remediation procedures which automatically detect and alert authorities about fatigued staff, track behavioral analysis with a timestamp, and warn users about potential risks. In this way, it is possible to provide effective bias free security and safety enforcement, assess when the issues are resolved, and assess a health of mobile and edge assets that are faulty. In other words, the assessments may be performed based on human behavior, physical hardware devices, and/or software components.

Aspects of the present invention may also allow a system integrator to enable an operations team with cognitive insights to build an augmented autonomous system to deliver zero-touch delivery, help proactively mitigate issues hosted on a multi-cloud hybrid environment extended with edge/internet of things (IOT) devices that lead to, for example, an exposure at audit and compliance. Aspects of the present invention may also provide recommendations to organize a secure approach that allows a level of access that needs to be established. For example, in embodiments, the recommendation may create a risk/opportunity analysis around policies to highlight their effect on performance and communication, which could include lost opportunities, reduction of valuable services, lack of production, and missed opportunities to increase efficiencies. Aspects of the present invention may further allow an original equipment manufacture (OEM)/partner eco-system recommendation to be involved at early stages in an engagement with information technology (IT) departments to develop comprehensive plans with OEMs that fit their production and security needs. The OEM/partner eco-system recommendation may help in understanding a cost of downtime and an amount of time that could be saved with the use of remote access.

According to an aspect of the invention, the system, method and computer program product uses contextual behavioral remediation and manage operations services in scalable critical mobile and edge applications. For example, the computer-implemented method includes: subscribing at least one user to a tracking system; proactively alerting a manager of the at least one user about at least one risk of the at least one user in the subscribed tracking system; providing auditable artifacts for safety and security compliance management; and combining emerging patterns between behavioral responses and at least one trait. In embodiments, the at least one trait may include genomic traits, skill level traits, experience traits, and regular physical activity traits. Further, patterns of the tracking system may be analyzed to provide insights on behavior response systems aiding medical support, categorization of people based on the at least one trait, and employing self-instructions, verbal instructions, exposure/response prevention, and stress coping strategy enhancement. The categorization of people may include people who are likely to handle certain kinds of problem statements better and utilize of skilled resources better.

Accordingly, implementations of the invention provide an improvement (i.e., technical solution) to a problem arising in the technical field of risk mitigation for occupational exposure. In addition, implementations of the invention provide an improvement (i.e., technical solution) to a problem arising in the technical field of enforcing a fatigue risk management plan and enabling transformation in worker behavior as part of user remediation, in addition to controlling many confounders, e.g., manipulating radiofrequency electromagnetic field (EMF) exposure.

It should be understood that, to the extent implementations of the invention collect, store, or employ personal information provided by, or obtained from, individuals (for example, health information), such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium or media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

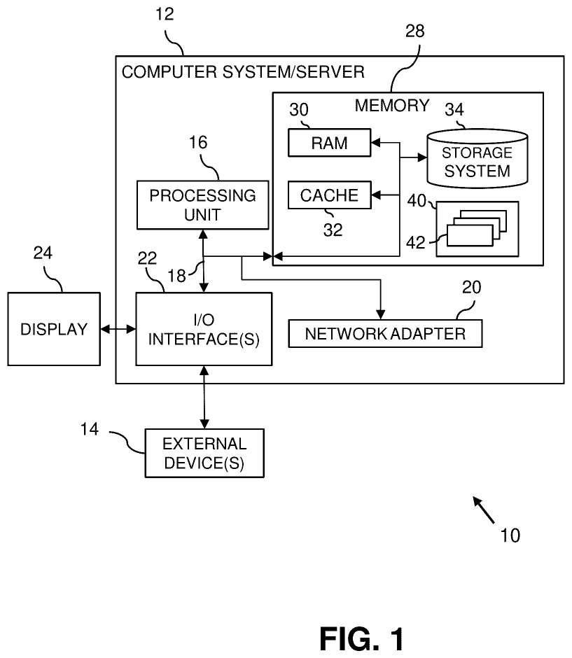

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

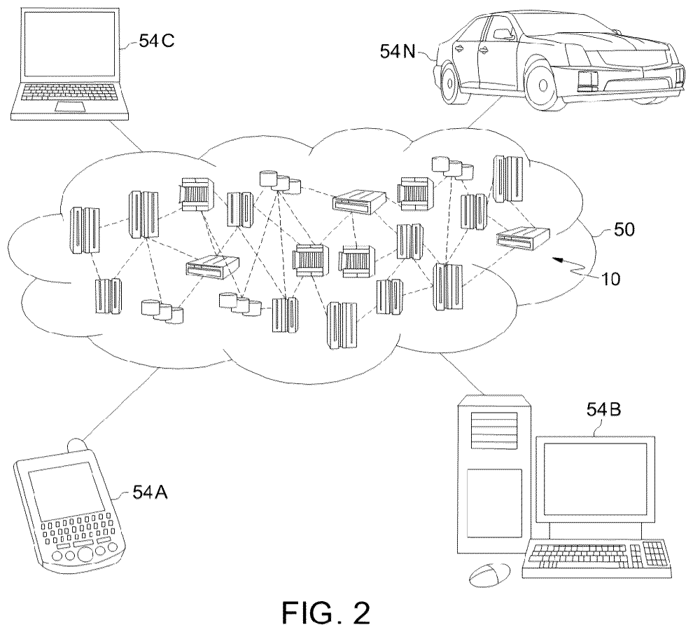

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

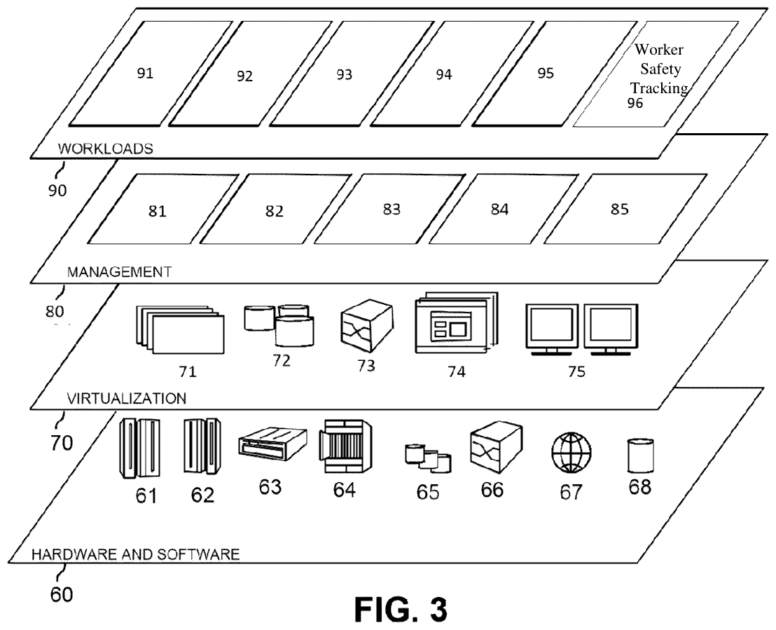

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and worker safety tracking 96.

Implementations of the invention may include a computer system/server 12 of FIG. 1 in which one or more of the program modules 42 are configured to perform (or cause the computer system/server 12 to perform) one of more functions of the worker safety tracking 96 of FIG. 3. For example, the one or more of the program modules 42 may be configured to: receive data which is associated with fatigue and/or exposure to a substance or radiation, for example; generate a personalized recommendation for the data by at least one artificial intelligence (AI) application; train a machine learning (ML) model using the data from the at least one AI application; and generate a trained fitness model for predicting safety issues based on the trained ML model using the data from the at least one AI application.

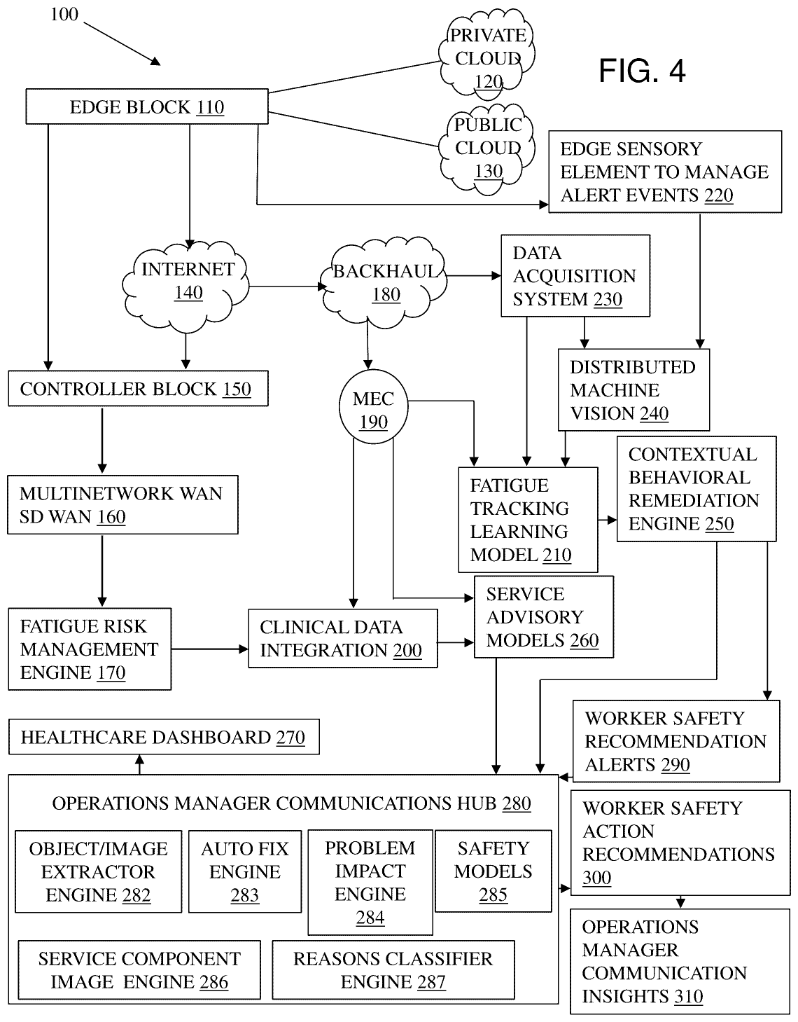

FIG. 4 shows a block diagram for fatigue identification and behavioral remediation in accordance with aspects of the invention. In embodiments, the block diagram 100 includes an edge block 110 which comprises a hardware device which comprises at least one of edge devices, edge clusters and gateways, network edge, and wireless networks. In particular, the edge block 110 may be used to enable a daily operational readiness test (DORT) objective to provide security insights and automate security and compliance by continuously monitoring and assessing risk across an edge environment of the edge block 100 and enforce governance. In embodiments, the edge block 110 may also communicate with at least one private cloud 120 and at least one public cloud 130 through one or more communication networks such as a local area network (LAN), wide area network (WAN), and the Internet, and communications thereof. The private cloud 120 and the public cloud 130 may be a multi-cloud management environment which has a distribution of cloud assets, software, and applications across several cloud hosting environments/providers in a single heterogenous architecture. The private cloud 120 and the public cloud 130 may be similar to the cloud computing environment 50 in FIG. 2

In FIG. 4, the edge block 110 communicates with a backhaul 180 through the Internet 140. Further, the edge block 100 also communicates with the controller block 150. The controller block 150 comprises software and/or hardware devices which includes software defined networking (SDN) multi-access edge computing (MEC) controller, a traffic monitor, a MEC resource and service monitoring database, and a provision planner. In this way, the controller block 150 may reduce a distance between resources of the edge block 110, computing resources and network resources in the block diagram 100. As an example, the traffic monitor of the controller block 150 may improve communication and reduce latency between the edge block 110 and the backhaul 180. Further, the MEC resource and service monitoring database of the controller block 150 may log information of a multi-network WAN software defined (SD) WAN 160. The multi-network WAN SD WAN 160 may use a redundancy model that relies on multiple cloud service providers to host an application in a single heterogenous architecture. The multi-network WAN SD WAN 160 may also be resilient, have regional availability, have performance and data sovereignty, have an integrated manageability, and security and visibility across multi-cloud deployments. The multi-network WAN SD WAN 160 may get a contextualized experience across applications and workloads.

In FIG. 4, the edge block 110 communicates with an edge sensory block 220 which comprises at least one hardware device. The edge sensory block 220 includes at least one edge sensory element to manage alert events. In particular, the at least one edge sensory element of the edge sensory block 220 determines a risk of alert services provided by a partner and/or vendor using a subscription process. The at least one edge sensory element of the edge sensory block 220 may compare the alert events and/or alert services with predetermined alerts to ensure that the alert events and/or services are not corrupted and/or defective. Further, the at least one edge sensory element of the edge sensory block 220 may monitor and assess risk and compliance objectives and enforce governance standards by determining whether the alert events and/or alerts services are correct and, for example, will improve the risk and compliance objectives and governance standards. Further, the at least one edge sensory element of the edge sensory block 220 may correct information that is identified as corrupted and/or defective and be proactive in implementing remediation steps.

In FIG. 4, the multi-network WAN SD WAN 160 connects to a fatigue risk management engine 170. The fatigue risk management engine 170 comprises a computing device including one or more elements of the computer system/server 12 of FIG. 1. The fatigue risk management engine 170 may alleviate and/or fix excess demand consumption, supplier delay, and divergence of parts handling, as examples. In particular, excess demand consumption relates to an excessive system load on the fatigue risk management engine 170. Supplier delay relates to a delay in a supply chain of devices by suppliers which are provided to the fatigue risk management engine 170. Divergence of parts handling relates to the way in which parts are handled by different workers and/or devices within the system of the block diagram 100. The fatigue risk management engine 170 may also provide contextual inventory risk insight by proactively identifying risk (e.g., situational and supplier attributes) and mitigation steps. In an example, the fatigue risk management engine 170 may keep track of a system load, a supplier delay, and parts handling, compare whether the system load, the supplier delay, and the parts handling are within predetermined ranges, and provide an alert with remediation steps when an inventory risk arises. Accordingly, the fatigue risk management engine 170 reduces the potential impacts of a revenue loss, recovery expenses, brand expenses and optimizes the expenses.

The backhaul 180 comprises at least one hardware device for connecting the edge block 110 to a clinical data integration 200 by using at least one multi-access edge computing (MEC) 190. The multi-access edge computing (MEC) 190 is a network hardware device that provides services and computing functions required by users on edge nodes.

In embodiments, the clinical data integration 200 comprises a computing device including one or more elements of the computer system/server 12 of FIG. 1. The clinical data integration 200 is configured to acquire clinical data from electronic health records, administrative records, claim records, patient/disease registries, etc. The clinical data integration 200 also comprises at least one hardware device (e.g., a plurality of sensors). The hardware device(s) may acquire analog measurements from the electronic health records, administrative records, claim records, patient/disease registries, other devices, etc., and use signal conditioning and an analog to digital conversion device to transform electrical waveforms of the analog measurements to a plurality of digital outputs.

The clinical data integration 200 communicates with service advisory models 260. The service advisory models 260 are configured for security management. The service advisory models 260 may comprise one or more program modules such as program modules 42 described with respect to FIG. 1. For example, the security management of the service advisory models 260 define an enterprise policy framework based on risk, security, and compliance (i.e., a role of a security/compliance officer), perform security controls and compliance management for a system, network, data, and/or audit readiness (i.e., a role of a security/compliance engineer). For example, the compliance management may implement policy based controls, operations visibility, collect evidence, and act based on alerts and notifications.

In embodiments, the backhaul 180 is also connected to a data acquisition system 230. The data acquisition system 230 comprises a process of data acquisition which entails acquiring sensor measurements from the backhaul 180 and processing an output to a human or a machine readable format.

Still referring to FIG. 4, a distributed machine vision 240 receives inputs from the data acquisition system 230 and the edge sensory block 220. The distributed machine vision 240 may be a software application at an edge and/or at an edge node cluster for receiving the inputs from the data acquisition system 230 and the edge sensory block 220 and forwarding this information to a fatigue tracking deep learning model 210. In particular, the distributed machine version 240 may comprise one or more program modules such as program modules 42 described with respect to FIG. 1.

The fatigue tracking deep learning model 210 comprises a computing device including one or more elements of the computer system/server 12 of FIG. 1. The fatigue tracking deep learning model 210 may provide real time data driven cognitive insights to combat fatigue using a sensory system and other connected devices (e.g., the MEC 190, the data acquisition system 230, and the distributed machine vision 140). The fatigue tracking deep learning model 210 may determine an operational fatigue risk, capture fatigue symptoms, assess incident reports, and determine an overall impact to the business. For example, the fatigue tracking deep learning model 210 may implement productivities and efficiencies (e.g., provide timely alerts, accurately track worker safety, and provide remediation options to improve worker safety) with regards to health issues, measure a risk attribute including a relationship and performance, assess a risk level which includes scheduled versus actual hours spent, history of an incident, and countermeasures, assess potential disruption events such as a conflict of interest, quality, lockdown, etc., assess potential impacts such as a rework, pay a premium for an emergency supply, reputation, etc., and assess preparation for audit and compliance.

The fatigue tracking deep learning model 210 may also provide insights on previous health patterns (e.g., physical patterns, mental patterns, job performance and family life matters) and false positive categories. The fatigue tracking deep learning model 210 may further assess the risk of alert services provided by clinical data by an internal subscription and provides a remediation technique. In particular, the fatigued tracking deep learning model 210 receives medical data, processes raw data and converts the raw data to a format that is consumed by artificial intelligence (AI) applications. The AI applications generate a personalized recommendation to limit worker safety issues (e.g., strenuous activity, fatigue, exposure to chemicals, etc.) and an alert, and then sends the converted data to a machine learning (ML) model. In an example, the AI applications may receive any combination of electrocardiogram (ECG) data, electromyography (EMG) data, galvanic skin response (GSR) data, foot data, hand data, and wearable devices (WD) data and, using this information, provide a personalized recommendation to limit strenuous activity to a predetermined time period within a workday based on the combination of received data. For example, an electrocardiogram (ECG) device, an electromyography (EMG) machine, a Geiger-muller (GM) detector, and chemical exposure monitoring (CEM) devices may be non-wearable devices, and may track one of a heart rate, muscle and nerve cell conditions, radiation, and exposure to chemicals.

The ML model trains the converted data and generates and/or updates a trained fitness model. In an example, the ML model may continuously receive any combination of ECG data, EMG data, GSR data, foot data, hand data, and WD data in order to generate and/or update the trained fitness model by determining a normal fatigue range, an above average fatigue range, and a below average fatigue range within an entire distribution of the combination of received data. In other words, the ML model may be trained to predict a fatigue level by comparing incoming data with the normal fatigue range, the above average fatigue range, and the below average fatigue range of historical data.

In embodiments, the fatigue tracking deep learning model 210 communicates with the contextual behavioral remediation engine 250. The contextual behavioral remediation engine 250 comprises a computing device including one or more elements of the computer system/server 12 of FIG. 1. The contextual behavioral remediation engine 250 is configured to take remediation action to improve worker safety (i.e., a role based on service owners and/or operations team) based on informed alerts and/or notifications by a recommended remediation when a user approval is required.

The contextual behavioral remediation engine 250 communicates with the worker safety recommendation alerts 290, which comprises a computing device including one or more elements of the computer system/server 12 of FIG. 1. In particular, the contextual behavioral remediation engine 250 receives medical data in the trained fitness model, determines whether there is fatigue based on the medical data, determines whether the fatigue has a duration greater than a predetermined threshold, and automatically alerts an authority when the fatigue has a duration greater than a predetermined threshold. The worker safety recommendation alerts 290 is configured to analyze all of the field issues, qualify the actions taken, restore critical services, and enable an incremental improvised knowledge set to derive an augmented service insight compared with service benchmarks.

In FIG. 4, the contextual behavioral remediation engine 250, the service advisory models 260, and the worker safety recommendation alerts 290 communicate with an operations manager communications hub 280. The operations manager communications hub 280 comprises a computing device including one or more elements of the computer system/server 12 of FIG. 1. In embodiments, the operations manager communications hub 280 comprises a digital dashboard which comprises a management console that presents data from multiple sources in a unified display which presents operational data in a way that is easier to read and interpret.

The operations manager communications hub 280 further comprises an object/image extractor engine 282, an auto fix engine 283, a problem impact engine 284, safety models 285, a service component image engine 286, and reasons classifier engine 287. The object/image extractor engine 282, the auto fix engine 283, the problem impact engine 284, the safety models 285, the service component image engine 286, and the reasons classifier engine 287 each comprise one or more program modules such as program modules 42 described with respect to FIG. 1.

In embodiments, the object/image extractor engine 282 provides prescriptive service instructions, and the auto fix engine 283 provides potential parts replacement to field engineers and optimizes the overall service cost. In particular, the prescriptive service instructions may include rule based service instructions to extract object and/or images received from the contextual behavioral remediation engine 250. The problem impact engine 284 and the service component image engine 286 interprets risk exposure and provides insights on a potential impact on an expected behavior of applications/infrastructure deviating from an actual design. In an example, the problem impact engine 284 and the service component image engine 286 may receive the enterprise policy framework and information regarding the security controls and compliance management for the system, network, data, and/or audit readiness from the service advisory models 260. With this information, the problem impact engine 284 and/or the service component image engine 286 may determine a risk exposure and a potential impact on the applications/infrastructure of the block diagram.

The reasons classifier engine 287 comprises a computing device to classify a reason to derive authentic information, derive a pattern from service tickets, source out clear reasons and map to standard error codes, control vague and unknown values and categorize false attributes/positives that can be used to manipulate data classification with utility functions and avoid bias. The operations manager communications hub 280 communicates with worker safety action recommendations 300. The worker safety action recommendations 300 comprise one or more program modules such as program modules 42 described with respect to FIG. 1. The safety models 285 and the worker safety action recommendations 300 enables predictive behavior to collaboration between the three actors and strengthening teamwork, healthcare environment, and a specific community. The safety models 285 and the worker safety action recommendations 300 also provides a cycle of safety for a patient practice staff, and a provider.

Still referring to FIG. 4, the operations manager communications hub 280 communicates with a healthcare dashboard 270. The healthcare dashboard 270 comprises a computing device including one or more elements of the computer system/server 12 of FIG. 1. Further, the healthcare dashboard 270 is configured to provide a unified digital experience to all process participant roles in a security management. In particular, the unified digital experience of the healthcare dashboard 270 provides a unified platform which digitally displays the tracking of worker safety, timely alerts provided based on the tracking of worker safety, and remediation steps to improve worker safety on a graphical user interface (GUI) within a display screen. Further, the healthcare dashboard 270 may simulate a number of roles including a security compliance officer, a security compliance engineer, and a service operations manager.

The worker safety action recommendations 300 communicates with the operations manager communication insights 310. The operations manager communication insights 310 comprises a computing device including one or more elements of the computer system/server 12 of FIG. 1. The operations manager communication insights 310 takes actions on informed alerts/notification by a recommended remediation where a user approval is required and may follow up with the user until the issue is resolved. In an example, the operations manager communication insights 310 may take actions on the informed alerts/notification by the recommended remediation and follow up with the user to determine whether the recommended remediation resolves the issue that prompted the informed alerts/notification. In a situation in which the issue is not resolved, the operations manager communication insights 310 may take another action with the user and follow up with the user to determine whether the other action resolves the issue. These steps may be repeated by the operations manager communication insights 310 until the issue is resolved.

The operations manager communication insights 310 may also send the insights to operators 1, 2, . . . , n (n is an integer) for a multimode live dialogue with all of the operators. In an example, the operations manager communication insights 310 may send the insights to operators 1 and 2 such that the operators 1 and 2 can participate in a multimode live dialogue to discuss the insights in real-time.

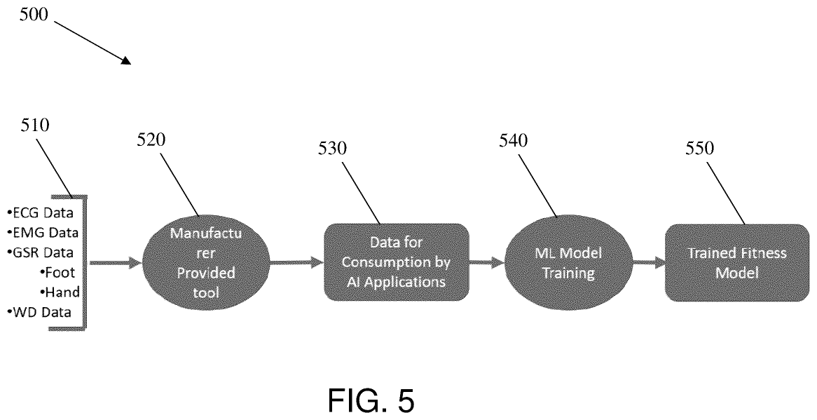

FIG. 5 shows a flowchart of a fatigue tracking deep learning model according to an embodiment of the present invention. Steps of the method may be carried out in the environment of FIG. 4 and are described with reference to elements depicted in FIG. 4. In the flowchart 500, at step 510, the system (e.g., fatigue tracking deep learning model 210 of FIG. 4) receives information including, for example, any combination of ECG data, EMG data, GSR data, foot data, hand data, and WD data, and passes the information to a manufacturer provided tool for processing raw data and converting the raw data to a format which can be used by artificial intelligence applications (AI) within the fatigue tracking deep learning model 210 at step 520. At step 530, the converted data is consumed by at least one artificial intelligence (AI) application and a personalized recommendation on at least one health risk and at least one timely alert on at least one health risk is generated within the fatigue tracking deep learning model 210. In an example, the AI applications may receive any combination of ECG data, EMG data, GSR data, foot data, hand data, and WD data and provide a personalized recommendation to limit worker safety issues (e.g., strenuous activity, fatigue, exposure to chemicals, etc.) to a predetermined time period within a workday based on the combination of received data. For example, an ECG device, an EMG machine, a Geiger-Muller (GM) detector, and chemical exposure monitoring (CEM) devices may be non-wearable devices and may track one of a heart rate, muscle and nerve cell conditions, radiation, and exposure to chemicals.

The converted data is input to a machine learning (ML) model for training at step 540. In an example, the ML model may continuously receive any combination of ECG data, EMG data, GSR data, foot data, hand data, and WD data in order to generate and/or update the trained fitness model by determining a normal fatigue range, an above average fatigue range, and a below average fatigue range within an entire distribution of the combination of received data. In other words, the ML model may be trained to predict a fatigue level by comparing incoming data with the normal fatigue range, the above average fatigue range, and the below average fatigue range of historical data.

In another example, the ML model may predict a fatigue level and then provide an alert for situations in which there is a continuous fatigue based on the converted data. In particular, the ML model receives GSR data of a user for different time intervals using at least one GSR sensor, determines whether the user is fatigued based on the GSR data of the user for the different time intervals, and detects a first conductance of a skin of the user in response to a determination that the user is fatigued in comparison to a second conductance of the skin of the user in response to a determination that the user is not fatigued. After the converted data is trained in the ML model, a trained fitness model is generated and/or updated with the trained data for use by the contextual behavioral remediation engine 250 at step 550.

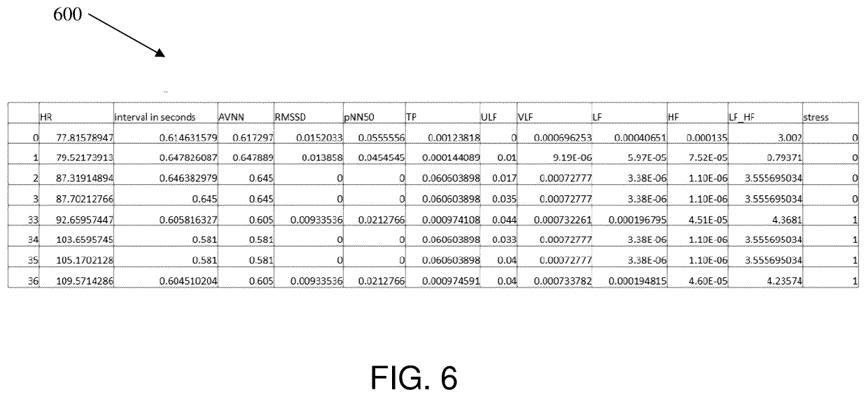

FIG. 6 shows sample model training data for the fatigue tracking deep learning model of FIG. 5 according to an embodiment of the present invention. In particular, the table 600 may include a heart rate (HR), an interval in seconds, average of all NN intervals (i.e., interval between two heartbeats), RMSSD (i.e., square root of the mean of the squares of differences between adjacent NN intervals), pNN50 (i.e., percentage of differences between adjacent NN intervals that are greater than 40 milliseconds), TP (i.e., total spectral power of all NN intervals up to 0.04 Hz), ULF (i.e., ultra low frequency), VLF (i.e., very low frequency), LF (i.e., low frequency), high frequency (i.e., HF), LF-HF (i.e., ratio of low frequency power to high frequency power; LF/HF), and stress (i.e., a binary value of either stress, 1 or no stress, 0). In the table 500, the stress is determined to be a “0” or “1” based on the model training data. The table 600 includes the information from the electrocardiogram (ECG) data, electromyography (EMG) data, galvanic skin response (GSR) data, foot data, hand data, and wearable devices (WD) data in the fatigue tracking deep learning model 210 of FIG. 4.

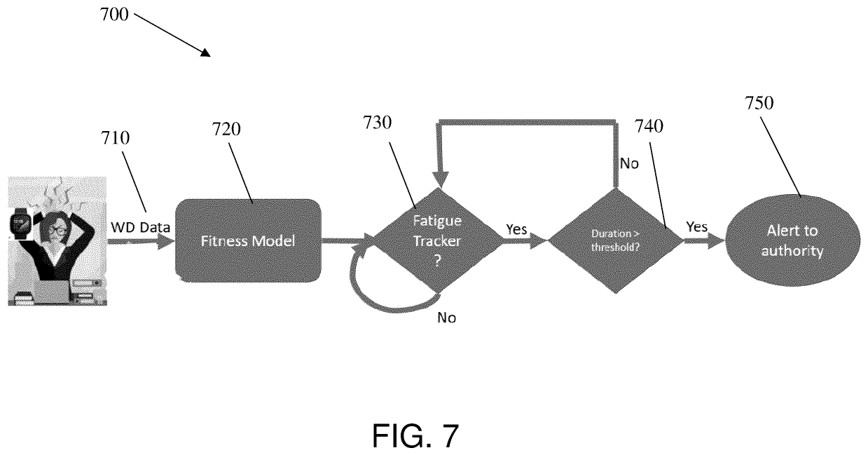

FIG. 7 shows a flowchart of fatigue identification in accordance with aspects of the invention. Steps of the method may be carried out in the environment of FIG. 4 and are described with reference to elements depicted in FIG. 4. In the flowchart 700, at step 710, wearable device (WD) data is collected from a wearable device on a user and the collected WD data is sent to the trained fitness model of the contextual behavioral remediation engine 250 shown in FIG. 4, at step 720. The WD data may continuously flow to the trained fitness model which predicts the fatigue level.

At step 730, the system (e.g., contextual behavioral remediation engine 250 of FIG. 4) determines whether a fatigue tracker is enabled. If the fatigue tracker is not enabled (i.e., “NO”), the method stops until the fatigue tracker is enabled. If the fatigue tracker is enabled (i.e., “YES”), the fatigue tracker determines whether there is fatigue based on the wearable device (WD) data and the trained fitness model. At step 740, the contextual behavioral remediation engine 250 determines whether fatigue has a duration greater than a predetermined threshold. If the fatigue has a duration less than the predetermined threshold (i.e., “NO”), the method loops back and performs fatigue tracking on additional WD data. If the fatigue has a duration greater than the predetermined threshold (i.e., “YES”), the method continues to step 750. At step 750, as the fatigue has a duration greater than the predetermined threshold, an authority is automatically alerted by the contextual behavioral remediation engine 250 (e.g., alert a manager, human resources, boss, etc.) In FIG. 7, the WD data comprises heart rate and heart rate variability captured using a photoplethysmography (PPG) sensor or an electrocardiogram (ECG) device, as an example. The heart rate and heart rate variability are determined by the PPG sensor measuring electrical signals based on light reflected from blood flow changes.

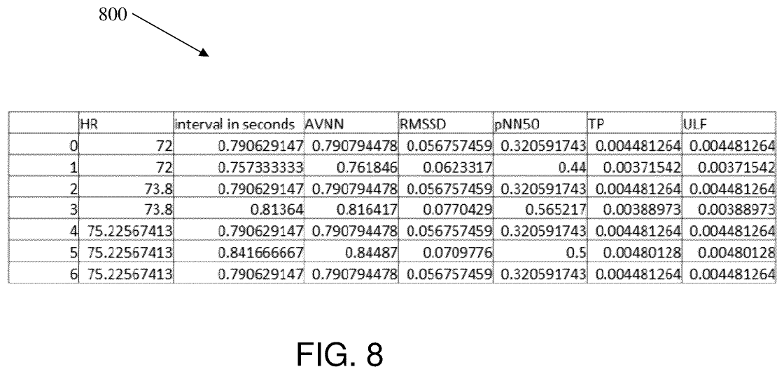

FIG. 8 shows sample wearable device data for the fatigue identification of FIG. 7 according to an embodiment of the present invention. In particular, the table 800 may include a heart rate (HR), an interval in seconds, average of all NN intervals (i.e., interval between two heartbeats), RMSSD (i.e., square root of the mean of the squares of differences between adjacent NN intervals), pNN50 (i.e., percentage of differences between adjacent NN intervals that are greater than 40 milliseconds), TP (i.e., total spectral power of all NN intervals up to 0.04 Hz), and ULF (i.e., ultra-low frequency) for the sample wearable device data for the fatigue identification.

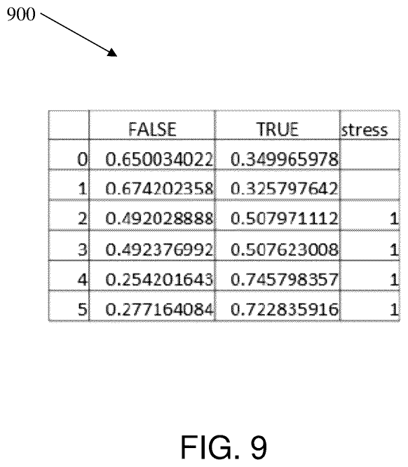

FIG. 9 shows a sample of predicted fatigue data for the fatigue identification of FIG. 7 according to an embodiment of the present invention. In particular, the table 900 includes data from the fatigue tracker of the contextual behavioral remediation engine 250 shown in FIG. 4. The table 900 shows probabilities of stress and predicts that the stress is “1” when the fatigue being true is above a 50% probability (i.e., fatigue is identified). If the fatigue being true is lower than a 50% probability, the stress is predicated to be “0” (i.e., no fatigue identification). The probabilities of stress may be calculated based on the fitness model which compares incoming medical data with the normal fatigue range, the above average fatigue range, and the below average fatigue range of historical data.

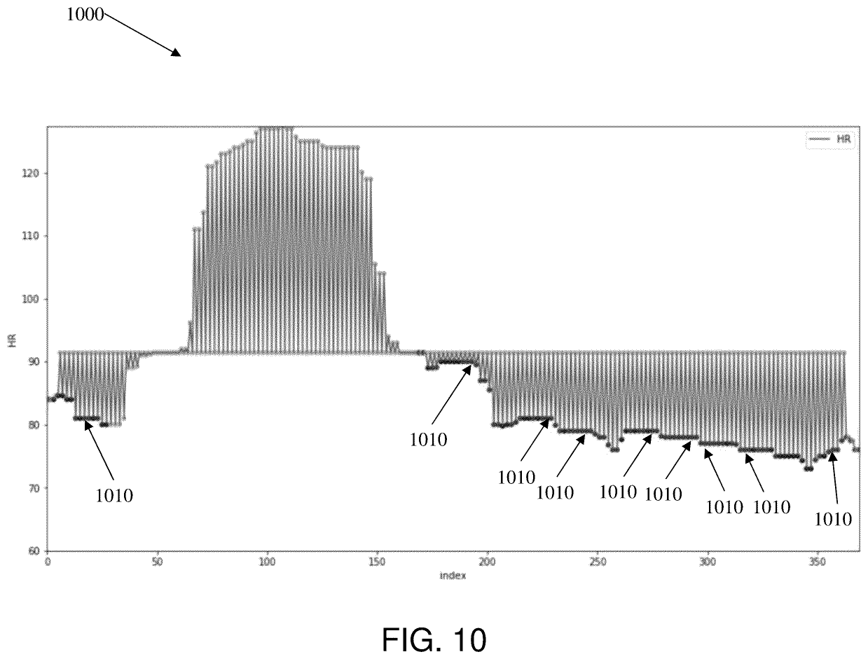

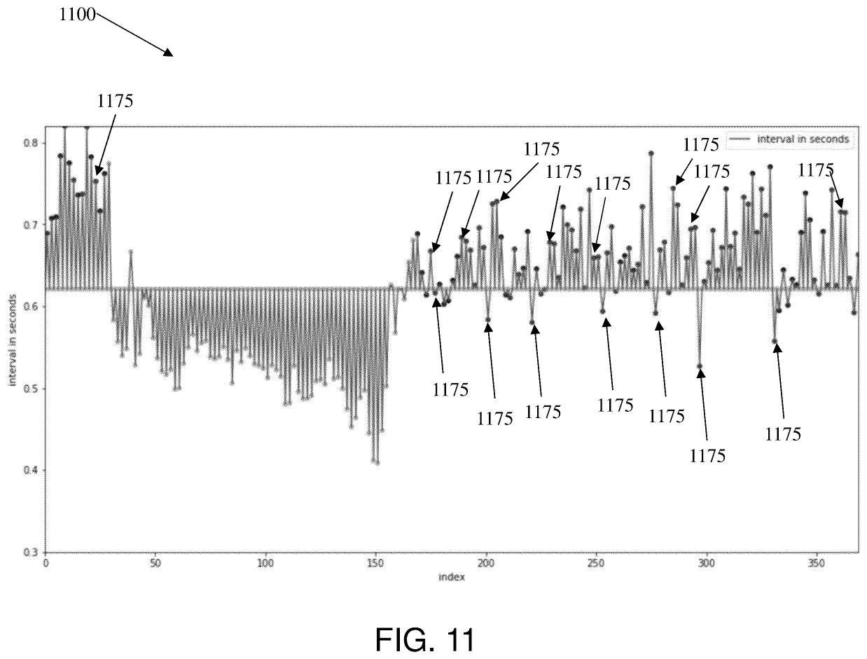

FIGS. 10 and 11 show graphs of predicted fatigue periods in accordance with aspects of the invention. In FIG. 10, in a graph 1000 of predicted fatigue periods, the x-axis is an index of information which may include ECG data, EMG data, GSR data, foot data, hand data, and WD data in FIGS. 5 and 6 and the y-axis is the heart rate (HR) of FIG. 6. In FIG. 10, the graph 1000 indicates flattening periods 1010 below a 90 HR in which fatigue is predicted. In FIG. 11, in a graph 1100 the x-axis in an index of information which may include ECG data, EMG data, GSR data, foot data, hand data, and WD data in FIGS. 5 and 6 and the y-axis is the interval in seconds of FIG. 6. In FIG. 11, the graph 1100 indicates points 1175 which indicate predicted fatigue periods.

In embodiments, a service provider could offer to perform the processes described herein. In this case, the service provider can create, maintain, deploy, support, etc., the computer infrastructure that performs the process steps of the invention for one or more customers. These customers may be, for example, a business that tracks worker safety and provides health remediation to improve worker safety. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system/server 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system/server 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving, by a computing device, data which is associated with a user;
generating, by the computing device, a personalized recommendation for the data by at least one artificial intelligence (AI) application;
training, by the computing device, a machine learning (ML) model using the data from the at least one AI application; and
generating, by the computing device, a trained fitness model which predicts safety issues based on the trained ML model using the data from the at least one AI application.

2. The method of claim 1, further comprising converting, by the computing device, the data into a format that is used by the at least one AI application.

3. The method of claim 1, wherein the data comprises at least one of electrocardiogram (ECG) data, electromyography (EMG) data, galvanic skin response (GSR) data, foot data, hand data, and wearable device (WD) data.

4. The method of claim 1, further comprising determining, by the computing device, whether there is user fatigue based on the data and the trained fitness model.

5. The method of claim 4, further comprising determining, by the computing device, whether a duration of the fatigue based on the data is greater than a predetermined threshold.

6. The method of claim 5, further comprising automatically providing, by the computing device, an alert in response to the fatigue being greater than the predetermined threshold.

7. The method of claim 6, wherein the data is wearable device (WD) data.

8. The method of claim 7, wherein the WD data comprises heart rate and heart rate variability.

9. The method of claim 8, wherein the heart rate and the heart rate variability are determined by a sensor which measures electrical signals based on light reflected from blood flow changes.

10. The method of claim 1, wherein the training the ML model further comprises:
receiving galvanic skin response (GSR) data of the user for different time intervals;
determining whether the user is fatigued based on the GSR data of the user for different time intervals; and
detecting a first conductance of a skin of the user in response to a determination that the user is fatigued in comparison to a second conductance of the skin of the user in response to a determination that the user is not fatigued,
wherein the second conductance of the skin of the user is different than the first conductance of the skin of the user.

11. The method of claim 1, wherein the personalized recommendation is based on a health risk of the data.

12. A computer program product comprising one or more computer readable storage media having program instructions collectively stored on the one or more computer readable storage media, the program instructions executable to:
receive, at a computing system, wearable device (WD) data of a user from a data acquisition system at an edge node cluster;
determine, at the computing system, whether the user has a safety issue based on the WD data by comparing the WD data and previous health pattern data;
determine, at the computing system, whether a duration of the safety issue based on the WD data is greater than a predetermined threshold in response to a determination that the user has the safety issue; and
automatically provide a worker safety recommendation alert, at the computing system, in response to a determination that the duration of the safety issue based on the WD data is greater than the predetermined threshold.

13. The computer program product of claim 12, wherein the wearable device (WD) data comprises medical data which comprises at least one of a heart rate, a time interval between heartbeats, and an ultra-low frequency.

14. The computer program product of claim 13, wherein the heart rate is determined by a sensor which measures electrical signals based on light reflected from blood flow changes of the user.

15. The computer program product of claim 12, wherein the alert is automatically provided to a manager of the user.

16. The computer program product of claim 12, wherein the safety issue is fatigue and the computing system comprises a fitness model which is trained to predict fatigue using historical WD data of the wearable device.

17. The computer program product of claim 16, wherein the fitness model is trained by using program instructions executable to:
receive galvanic skin response (GSR) data of the user for different time intervals using at least one GSR sensor;
determine whether the user is fatigued based on the GSR data of the user for different time intervals; and
detect a first conductance of a skin of the user in response to a determination that the user is fatigued in comparison to a second conductance of the skin of the user in response to a determination that the user is not fatigued,
wherein the second conductance of the skin of the user is different than the first conductance of the skin of the user.

18. A system comprising:
a processor, a computer readable memory, one or more computer readable storage media, and program instructions collectively stored on the one or more computer readable storage media, the program instructions executable to:
receive data associated with fatigue at an edge node cluster;
generate a personalized recommendation to limit worker safety issues for the data associated with the fatigue by at least one artificial intelligence (AI) application;
train a machine learning (ML) model using the data from at least one AI application and historical information associated with the data; and
generate a trained fitness model which predicts safety issues based on the trained ML model using the data from the at least one AI application and the historical information associated with the data.

19. The system of claim 18, wherein the data includes at least one of electrocardiogram (ECG) data, electromyography (EMG) data, galvanic skin response (GSR) data, foot data, hand data, and wearable device (WD) data.

20. The system of claim 18, wherein the training the ML model using the data further comprises program instructions executable to:
receive galvanic skin response (GSR) data of a user for different time intervals using at least one GSR sensor;
determine whether the user is fatigued based on the GSR data of the user for different time intervals; and
detect a first conductance of a skin of the user in response to a determination that the user is fatigued in comparison to a second conductance of the skin of the user in response to a determination that the user is not fatigued,
wherein the second conductance of the skin of the user is different than the first conductance of the user.
